(19) United States
(12) Patent Application Publication
(10) Pub. No.: US 2009/0112266 A1
(43) Pub. Date: Apr. 30, 2009

(54) SPINAL DYNAMIC STABILIZATION DEVICE

(75) Inventors: Yu Shih Weng, Pingtung County (TW); Chia-Wei Yu, Taipei City (TW); Yi-Hung Lin, Hsinchu County (TW); I-Ching Wu, San Francisco, CA (US); Ya-Jen Yu, Taipei City (TW); Wen-Jer Chen, Taipei City (TW); Shan-Chang Chueh, Shihlin District (TW); Chris Ing-Yi Huang, Hsinchu (TW); Jaw-Lin Wang, Taipei City (TW); Shian-Yih Wang, Taipei (TW)

Correspondence Address: QUINTERO LAW OFFICE, PC, 2210 MAIN STREET, SUITE 200, SANTA MONICA, CA, 90405, US

(73) Assignee: INDUSTRIAL TECHNOLOGY RESEARCH INSTITUTE, HSINCHU (TW)

(21) Appl. No.: 12/257,818

(22) Filed: Oct. 24, 2008

(30) Foreign Application Priority Data
Oct. 25, 2007 (TW) TW096140021

(51) Int. Cl. A61B 17/70; A61B 17/04

(52) U.S. Cl. 606/257; 606/246; 606/300

(57) ABSTRACT

A spinal dynamic stabilization device for maintaining an anatomical height between two adjacent vertebras is provided. Each vertebra includes a spinous process and two symmetric pedicles. The spinal dynamic stabilization device includes a supporting member, at least one anchoring member, and at least one connecting member. The supporting member is disposed between the spinous processes. The anchoring member is fixed in one of the vertebra via one of the pedicles. The connecting member connects the supporting member to the anchoring member, fixing a relative position between the supporting member and the anchoring member, further fixing a relative position between the vertebras.

### CROSS REFERENCE TO RELATED APPLICATIONS

This Application claims priority of Taiwan Patent Application No. 096140021, filed on Oct. 25, 2007, the entirety of which is incorporated by reference herein.

### BACKGROUND OF THE INVENTION

1. Field of the Invention

The invention relates to a spinal dynamic stabilization device, and more particularly to a spinal dynamic stabilization device which can correct the normal height between vertebrae of the spine.

2. Description of the Related Art

At present, spondylolisthesis and spinal stenosis are common diseases. Spondylolisthesis usually occurs in workers whom require constant lifting of heavy items and athletes, and is caused by pressure and extra force on the disc. Namely, one of the vertebra of the vertebral column slides forward to a neighboring vertebra. At which time, the vertebra presses central nerves or nerve roots and the patient feels pain. Stenosis is the narrowing of the spinal canal, pathologically caused by aging. For stenosis, central nerves or nerve roots in the spinal canal are compressed, resulting in lower back pain for patient.

To cure the above-mentioned diseases, spinal fusion is usually performed. However, spine non-fusion may eventually develop due to limited patient movement degenerating adjacent discs due to overweight pressure.

U.S. Pat. No. 5,609,635 discloses a spinal fusion implant embedded between adjacent vertebras for replacing a degenerative disc. And then autologous bone is filled in the inner of the spinal fusion implant. Biological activity from autologous bone induces bone growing between vertebras for spinal fusion.

U.S. Pat. No. 7,083,622 discloses a facet screw fixed on a facet joint. Vertebras under and above the facet joint are fixed via screws. In the vertical and horizontal direction, a spinal implant rod is connected to a connector, and a movable sliding device is disposed therebetween for adjustment as implantation.

U.S. Pat. No. 5,282,863 discloses a flexible stabilization system to fix the middle of one vertebra and an adjacent vertebra via stabilization elements and screws. The stabilization elements and screws are made of nonmetal with durability, bio-compatibility, and flexibility to provide space for the spine to move.

U.S. Pat. No. 6,770,075 discloses a spinal fixation apparatus comprising anchor screws, a rod and a spacer. The anchor screws are respectively fixed to the sides of one vertebra and adjacent up and down vertebras. The anchor screws are connected in series via the rod. The spacer surrounds the rod but permits the vertebras to rotate in predetermined angles and to move. Thus, the dislocated vertebra is restored to the physiological normal state.

U.S. Pat. No. 7,074,237 discloses an element similar to a yoke line with a hole for containing a screw, fixed on two sides of a vertebra. The bottom of the element similar to a yoke line may be disposed between adjacent spinous processes for maintaining height of the vertebras.

U.S. Pat. No. 5,645,599 discloses a U-shaped body, embedded between adjacent two spinous processes, of which the sides respectively comprises a bracket protruding upward for engaging with the spinous processes. An elastic body is installed on the inner of the U-shaped body for cushion.

U.S. Pat. No. 6,068,630 discloses a spine distraction implant disposed between adjacent two spinous processes, for maintaining physiological height between the spinous processes. Moreover, the spine distraction implant comprises a wing portion on the front end and the rear end, protruding outward and attaching to two sides of the spinous processes for fastening.

### BRIEF SUMMARY OF THE INVENTION

The invention provides a spinal dynamic stabilization device to reduce complexity and time of operation, to restore height between two adjacent vertebras, to increase dynamic stability between vertebrae, to mitigate nerve compression caused by degenerative spondylolisthesis and spinal stenosis of the spinal canal, and to ease a patient's pain.

The invention is adapted to maintain height between two adjacent vertebras, wherein each vertebra comprises a spinous process (a single member protrudes backward and downward which is the attaching point of soft tissue), transverse process (a pair of member which is the attaching point of soft tissue), a pedicle and a vertebral body. The spinal dynamic stabilization device comprises: a supporting member disposed between the spinous processes; at least one anchoring member fixed in one of the vertebra via one of the pedicles; and at least one connecting member connecting the supporting member to the anchoring member, fixing a relative position between the supporting member and the anchoring member, further fixing a relative position between the vertebrae.

The connecting member, the supporting member, and the anchoring member relatively move and comprise at least a movable connecting point for increasing dynamic stability.

According to the spinal dynamic stabilization device of the invention, the supporting member is made of an elastic material or is assembled by an elastic mechanism.

The structure of the supporting member comprises a concentric circular structure, a mesh structure, a multi-layered structure, a radiate structure, or an artificial disc structure.

The structure of the supporting member comprises a hollow pillared structure, porous structure, a sponge structure, a multi-layered structure, a filled structure or an assembled structure.

The supporting member is made of a bio-compatible material, a porous material, a multi-layered material, a shape memory material or a damping material.

The connecting member is detachably connected to the supporting member.

The connecting member and the supporting member are a single and unitary member.

The supporting member comprises a spherical groove, the connecting member comprises a spherical member, and the spherical member is rotatably disposed in the spherical groove.

The supporting member comprises a depression, the connecting member comprises a protrusion, and the protrusion is engaged with the depression.

The supporting member comprises a groove and a screw hole, the connecting member comprises a connecting end and an opening, the screw hole adjoins the groove, the opening is disposed on the connecting end, the connecting end is disposed in the groove, the screw is adjusted and aligned according to the opening, and the connecting end is disposed in the opening via a screw and locked in the screw hole to fix the connecting end in the groove.

The supporting member comprises a protrusion, the connecting member comprises a loop, and the loop is hooked onto the protrusion.

The spinal dynamic stabilization device further comprises a clamp to fix and connect to the supporting member and the connecting member, wherein the clamp comprises a clipping groove and a clipping hole, the clipping groove is disposed on the outer side of the clipping hole, the connecting member is movably clipped by the clipping groove, the supporting member is held in the clipping hole.

The connecting member is made of a rigid material, an elastic material, or a viscoelastic material, and the connecting member is assembled by a rigid mechanism, an elastic mechanism or a viscoelastic mechanism.

The connecting member is linear shaped, pillared shaped, plate shaped, curve shaped or spring shaped.

The connecting member is made of a bio-compatible material, a porous material, a multi-layered material, a shape memory material or a damping material.

The connecting member is detachably connected to the anchoring member.

The anchoring member comprises a pedicle screw and is fixed in one of the vertebras.

The anchoring member is fixed in one of the vertebras via Polymethylmethacrylate.

The anchoring member is made of a bio-compatible material.

The anchoring member comprises a depression and a locking portion, the locking portion is installed adjacent to the depression, and the connecting member extends to the depression and is fixed in the anchoring member via the locking portion.

The connecting member comprises a loop, and the loop is hooked onto the anchoring member.

The anchoring member comprises a depression, the connecting member comprises an engaging portion, and the engaging portion is engaged with the depression.

The shapes of depression and the engaging portion are complementary.

The anchoring member comprises at least a groove, the groove is engaged with at least a connecting member or at least a supporting member, and the anchoring member comprises an inner groove for inward fixing the connecting member or the supporting member.

The anchoring member comprises a lateral offset to fix to the connecting member, and the lateral offset is a joint.

The head of the anchoring member is a joint.

### BRIEF DESCRIPTION OF DRAWINGS

The invention can be more fully understood by reading the subsequent detailed description and examples with references made to the accompanying drawings, wherein:

FIG. 1 is a schematic view of an embodiment of a spinal dynamic stabilization device;

FIG. 2 is a schematic view of an embodiment of an anchoring member of a spinal dynamic stabilization device;

FIG. 3 is a schematic view showing a spine and a tissue speculum;

FIG. 4 is a schematic view showing a supporting member of a spinal dynamic stabilization device combined with a spine;

FIG. 5 is a cross-sectional of a vertebrae, wherein an anchoring member of a spinal dynamic stabilization device is fixed in the vertebrae;

FIG. 6 is a schematic view showing a spinal dynamic stabilization device combined with a spine;

FIGS. 7A, 7B, 7C, 7D and 7E are schematic views of an embodiment of a combined connecting member and supporting member of a spinal dynamic stabilization device;

FIGS. 8A and 8B are schematic views showing an embodiment of a combined anchoring member and vertebrae of a spinal dynamic stabilization device;

FIGS. 9A and 9B are schematic views showing an embodiment of a combined connecting member and anchoring member of a spinal dynamic stabilization device;

FIG. 10 is a schematic view of another embodiment of a spinal dynamic stabilization device;

FIG. 11 is a schematic view showing another embodiment of a spinal dynamic stabilization device combined with a spine;

FIGS. 12A and 12B are schematic views showing another embodiment of a spinal dynamic stabilization device combined with a spine, wherein a connecting member may be a rope-shaped member or a spring assembled member with a speculum;

FIGS. 13A, 13B and 13C are schematic views showing another embodiment of a combined connecting member and anchoring member of a spinal dynamic stabilization device;

FIGS. 14A and 14B are schematic views of another embodiment of a connecting member of a spinal dynamic stabilization device;

FIG. 15A is a schematic view of another embodiment of a connecting member and a clamp of a spinal dynamic stabilization device;

FIG. 15B is a cross-sectional view along a cross-sectional line A-A;

FIG. 16 is a schematic view showing another embodiment of a spinal dynamic stabilization device combined with a spine; and

FIG. 17 is a schematic view showing another embodiment of a spinal dynamic stabilization device combined with a spine.

The invention can be more fully understood by reading the subsequent detailed description and examples with references made to the accompanying drawings.

### DETAILED DESCRIPTION OF THE INVENTION

### First Embodiment

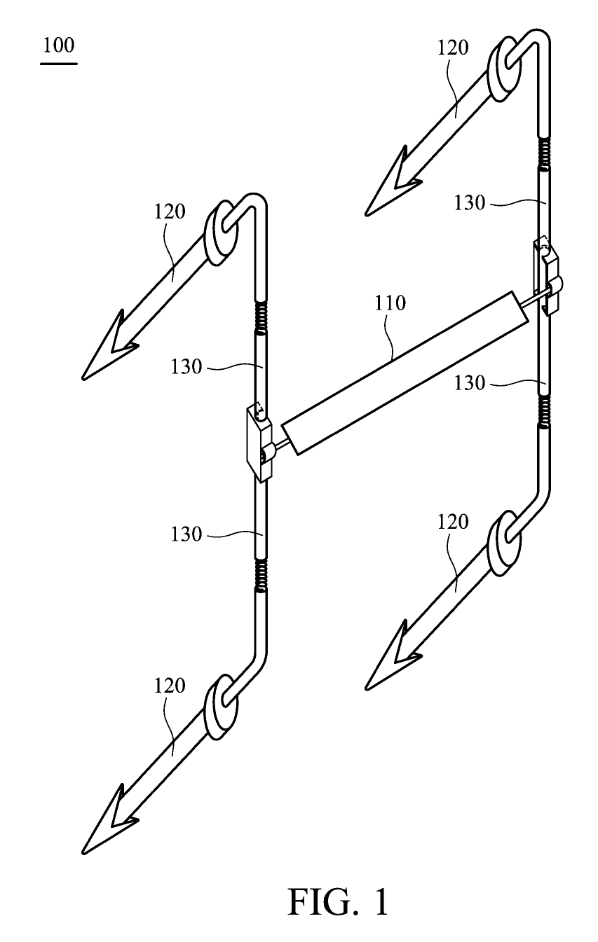

Referring to FIG. 1, a spinal dynamic stabilization device 100 comprises a supporting member 110, four anchoring members 120 and four connecting members 130.

The supporting member 110 is made of a bio-compatible material, a porous material, a multi-layered material, a shape memory material or a damping material. If the supporting member 110 is made of an elastic material or is assembled by an elastic mechanism, the structure thereof comprises a concentric circular structure, a mesh structure, a multi-layered structure, a radiate structure, or an artificial disc structure. The structure of the supporting member 110 comprises a hollow pillared structure, porous structure, a sponge structure, a multi-layered structure, a filled structure or an assembled structure. The supporting member 110 is connected to the connecting member 130 by virtue of a clamp 150 for fixing or maintaining dynamic connection. Dynamic connection is defined as the supporting member 110 and the connecting member 130 being completely fixed, one end of the supporting member 110 and the connecting member 130 able to move, or two ends of the supporting member 110 and the connecting member 130 able to move.

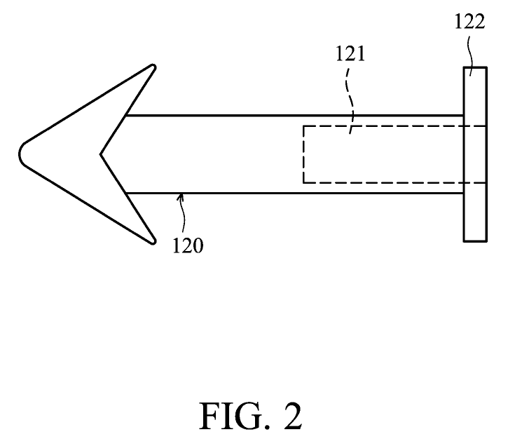

Referring to FIG. 2, each anchoring member 120 comprises a depression 121 and a locking portion 122. The locking portion 122 is installed adjacent to the depression 121. Each anchoring member 120 is a pedicle screw and is made of a bio-compatible material.

Referring to FIG. 1, each connecting member 130 is connected to the supporting member 110 and the anchoring member 120 for maintaining the relative position between the supporting member 110 and the anchoring member 120. Note that each connecting member 130 is detachably connected to the supporting member 110 or each connecting member 130 and the supporting member 110 are a single and unitary member. Each connecting member 130 is detachably connected to the anchoring member 120. To illustrate in detail, referring to FIGS. 1 and 2, each connecting member 130 protrudes to the depression 121 of the anchoring member 120. Each connecting member 130 is fixed to each anchoring member 120 via the locking portion 122. The connecting member 130 is made of a rigid material, an elastic material, or a viscoelastic material, and the connecting member is assembled by a rigid mechanism, an elastic mechanism or a viscoelastic mechanism. The connecting member 130 is made of a bio-compatible material. The material comprises a porous material, a multi-layered material, a shape memory material or a damping material. The connecting member 130 is linear shaped, pillared shaped, plate shaped, or curve shaped. The connecting member 130 is connected to the anchoring member 120 for completely fixing or maintaining dynamic connection. The dynamic connection is defined as the connecting member 130 and the anchoring member 120 being completely fixed, one end of the connecting member 130 and the anchoring member 120 able to move, two ends of the connecting member 130 and the anchoring member 120 able to move, three ends of the connecting member 130 and the anchoring member 120 able to move or four ends of the connecting member 130 and the anchoring member 120 able to move.

Following, the corrective therapy using the spinal dynamic stabilization device 100 for correcting degenerative spondylolisthesis and spinal stenosis and correcting the normal height between vertebras of the spine is described.

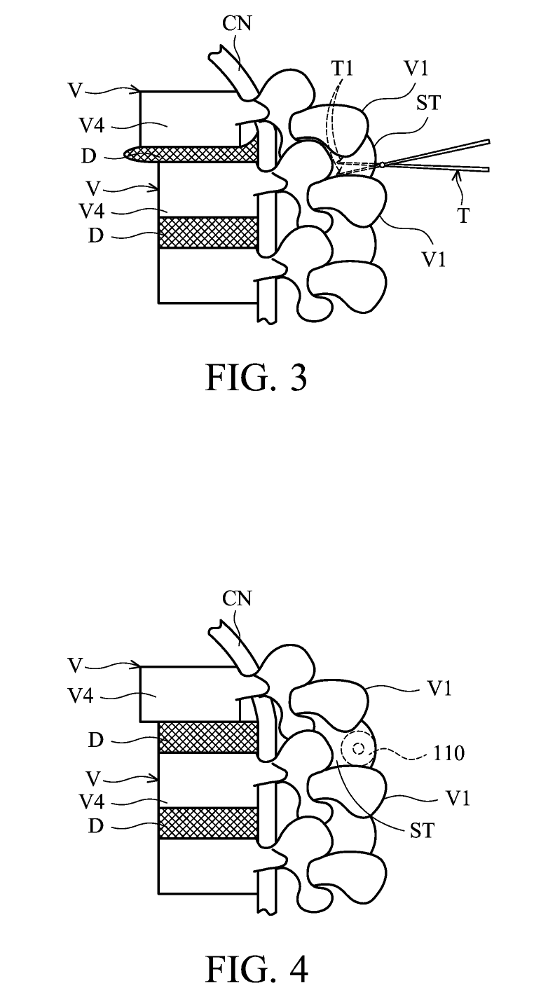

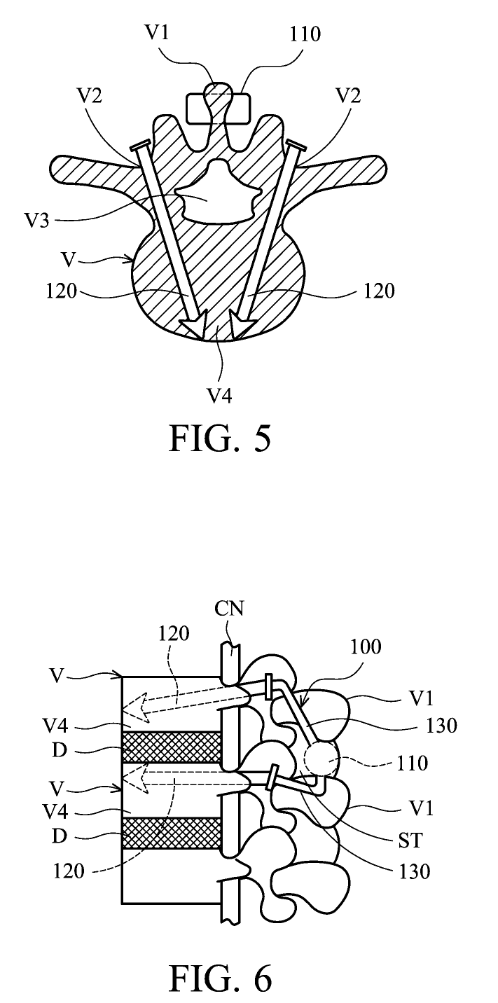

Referring to FIG. 3, the soft tissue ST is punctured and braced between two adjacent vertebras V via two tip ends T1 of a tissue expender. Referring to FIGS. 3 and 5, each vertebra V comprises a spinous process V1, two symmetrical vertebras V2, a vertebral cavity V3 and a vertebral body V4. A central nerve CN passes and is disposed in the vertebral cavity V3. The disc D is disposed between two adjacent vertebra bodies V4 and the soft tissue ST is disposed between two adjacent spinous processes V1. Referring to FIG. 4, the supporting member 110 is disposed in the braced and open soft tissue ST. At the same time, the supporting member 110 is propped between the two adjacent spinous processes V1 and separates the adjacent spinous processes V1 (or vertebra V). Referring to FIG. 5, two anchoring members 120 are respectively installed on the vertebras V2 of the vertebra V that slide forward. The anchoring members 120 relatively disposed above the vertebras V2 are fixed in (the vertebra bodies V4 of) the vertebra V sliding forward. The anchoring members 120 relatively disposed under the vertebras V2 are fixed in (another vertebra bodies V4 of) another vertebra V adjacent to the vertebra V sliding forward for maintaining the relative position between the vertebra V which slid forward and another adjacent vertebra V. The anchoring members 120 is fixed in (the vertebra bodies V4 of) the vertebra V via outer thread. Finally, referring to FIG. 6, each connecting member 130 is respectively connected between the supporting member 110 and each anchoring members 120 for pulling back the vertebra V which slides forward and maintain the relative position between the vertebra V and another adjacent vertebra V. The four connecting members 130 respectively comprise a suitable length for pulling the vertebra V which slides forward back a normal biological position. As such, the original height between the vertebra V is effectively restored and dynamic stability increases.

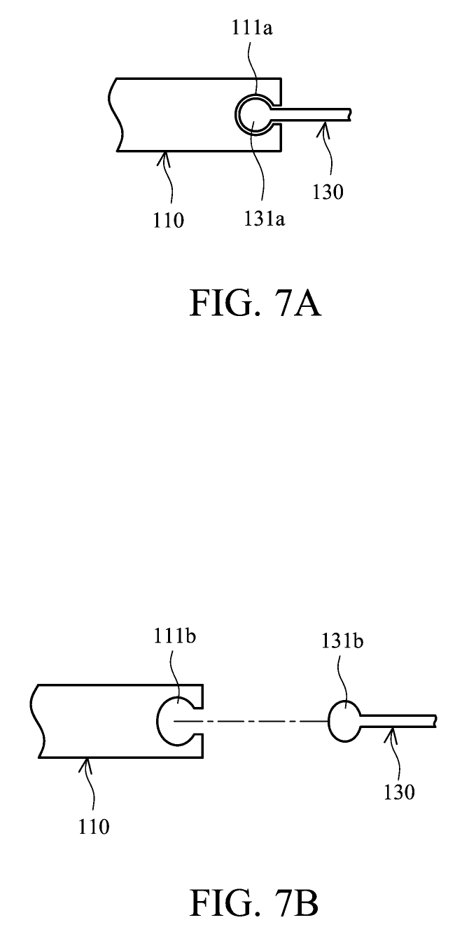

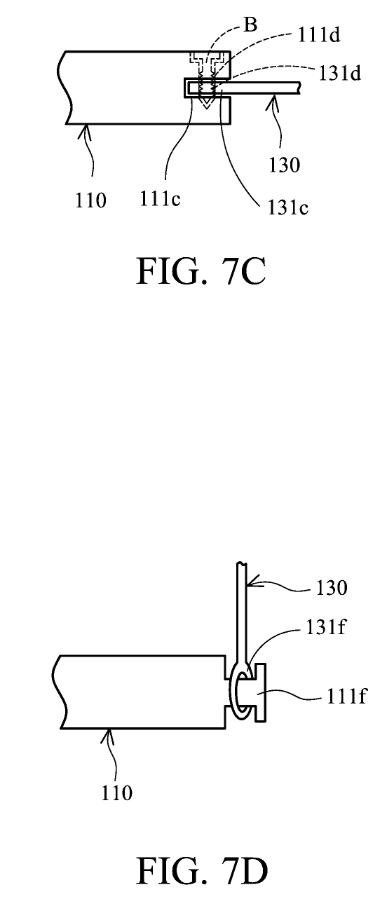

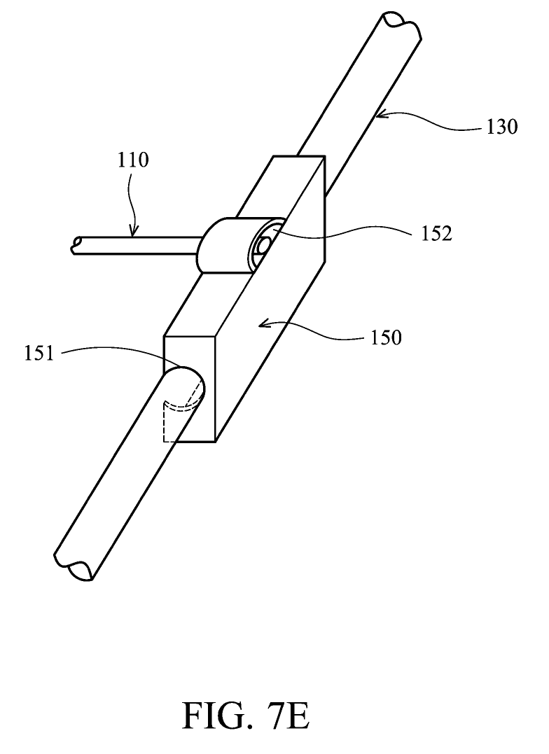

Each connecting member 130 is connected to the supporting member 110 by various methods. For example, referring to FIG. 7A, the supporting member 110 comprises a spherical groove 111a. Each connecting member 130 comprises a spherical member 131a. When each connecting member 130 is connected to the supporting member 110, the spherical member 131a of the connecting member 130 is rotatably installed in the spherical groove 111a of the supporting member 110. Referring to FIG. 7B, the supporting member 110 comprises a depression 111b, and each connecting member 130 comprises a protrusion 131b. When the connecting member 130 is connected to the supporting member 110, the protrusion 131b of each connecting member 130 is engaged with the depression 111b of the supporting member 110. Referring to FIG. 7C, the supporting member 110 comprises a groove 111c and a screw hole 111d. Each connecting member 130 comprises a connecting end 131c and an opening 131d. The screw hole hid adjoins the groove 111c. The opening 131d is disposed on the connecting end 131c. When each connecting member 130 is connected to the supporting member 110, the connecting end 131c of each connecting member 130 is installed in the groove 111c of the supporting member 110. The opening 131d is adjusted and aligned according to the screw hole 111d, and the connecting end 131c is disposed in the opening 131d via a screw B and locked in the screw hole hid to fix the connecting end 131c in the groove 111c. Referring to FIG. 7D, the supporting member 110 comprises a protrusion 111f, and each connecting member 130 comprises a loop 131f. When each connecting member 130 is connected to the supporting member 110, the loop 131f of each connecting member 130 hooks the protrusion 111f of the supporting member 110. Referring to FIG. 7E, the spinal dynamic stabilization device 100 comprises a clamp 150 to fix and connect to the supporting member 110 and the connecting member 130. In detail, the clamp 150 comprises a clipping groove 151 and a clipping hole 152. The clipping groove 151 is disposed on the outer side of the clipping hole 152. The clipping groove 151 movably clips the connecting member 130. The supporting member 110 is held in the clipping hole 152. The connecting member 130 and the supporting member form an included angle.

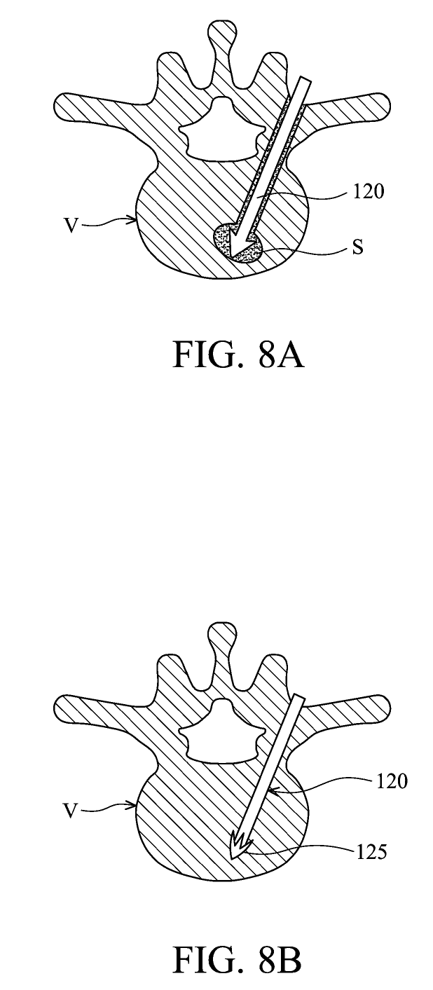

Each anchoring member 120 is fixed in the vertebras V via various methods. For example, referring to FIG. 8A, each anchoring members 120 is fixed in the vertebras V via Polymethylmethacrylate (PMMA). Referring to FIG. 8B, each anchoring members 120 comprises a radiate hook 125. When each anchoring members 120 is fixed in the vertebras V, the radiate hook is engaged in one of the vertebras V.

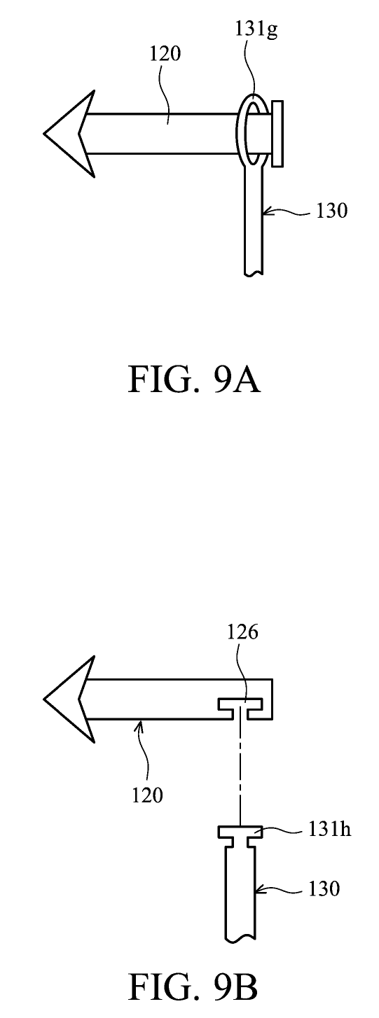

Each connecting member 130 is fixed to each anchoring member 120 via various methods. For example, Referring to FIG. 9A, each connecting member comprises a loop 131g, and the loop 131g is hooked on the anchoring member 120. Referring to FIG. 9B, each anchoring member 120 comprises a depression 126. Each connecting member 130 comprises an engaging portion 131h. The shapes of depression 126 and the engaging portion 131h are complementary. When each connecting member 130 is connected to each anchoring member 120, the engaging portion 131h is engaged with the depression 126 to provide the depression 126 and engaging portion 131h with partial movement.

### Second Embodiment

In another embodiment, the elements which are the same as the above-mentioned embodiment are labeled with the same number.

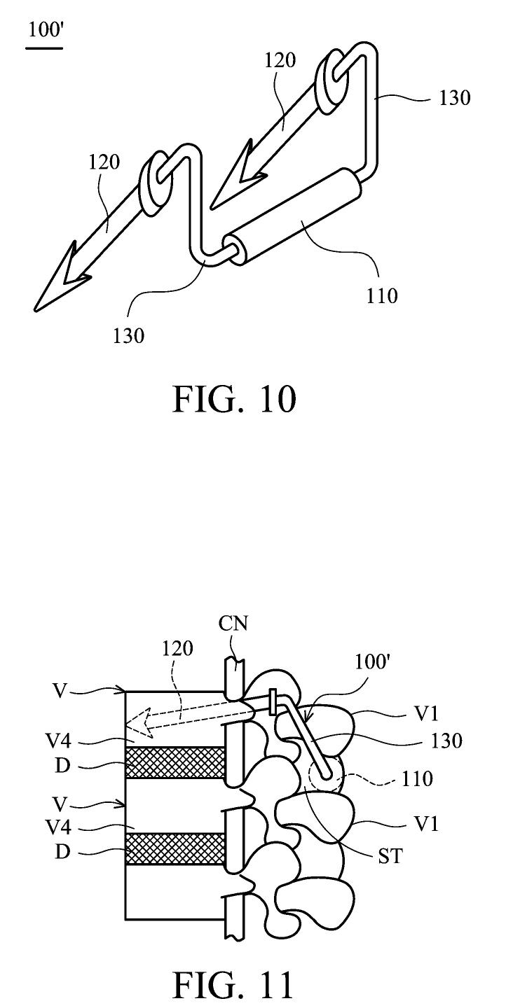

Referring to FIG. 10, the spinal dynamic stabilization device 100′ comprises a supporting member 110, two anchoring members 120 and two connecting members 130.

Similarly, each connecting member 130 connects the supporting member 110 to the anchoring member 120, fixing a relative position between the supporting member 110 and the anchoring member 120. The connecting member 130 is connected to the anchoring member 120 via complete fixation or dynamic connection. Dynamic connection is defined as having one end of the supporting member 110 and the connecting member 130 able to move, or two ends of the supporting member 110 and the connecting member 130 able to move.

Other element structures, characteristics and operating methods of this embodiment that are the same as the above-mentioned embodiment are omitted for brevity.

### Third Embodiment

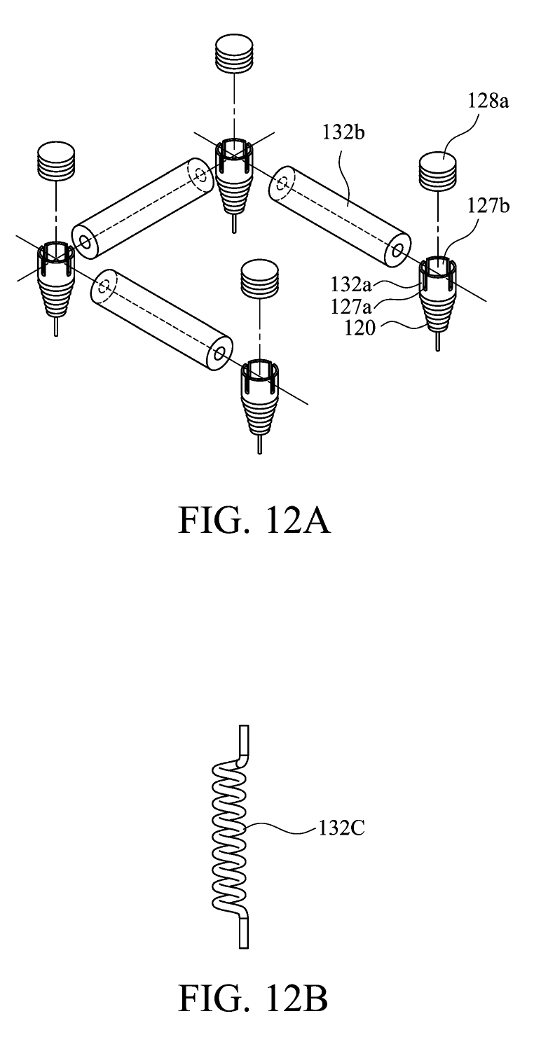

Referring to FIG. 12A, the anchoring member 120 is fixed in the vertebra V. At least a groove 127a is installed above the anchoring member 120. The groove 127a is engaged with at least a connecting member 130 or supporting member 110. The anchoring member 120 comprises an inner groove 127b to fix to an inner screw 128a for fixing the connecting member 130. The connecting member 130 is made of a rigid material, an elastic material, or a viscoelastic material, and the connecting member is assembled by a rigid mechanism, an elastic mechanism or a viscoelastic mechanism. The connecting member 130 or the supporting member 110 may be a rope-shaped member, assembled member with a supporting member 132b (the shape may be sleeve-shaped, pillared shaped, plate shaped, or other shaped) (shown in FIG. 12A), a spring 132c or a viscoelastic mechanism (shown in FIG. 12B).

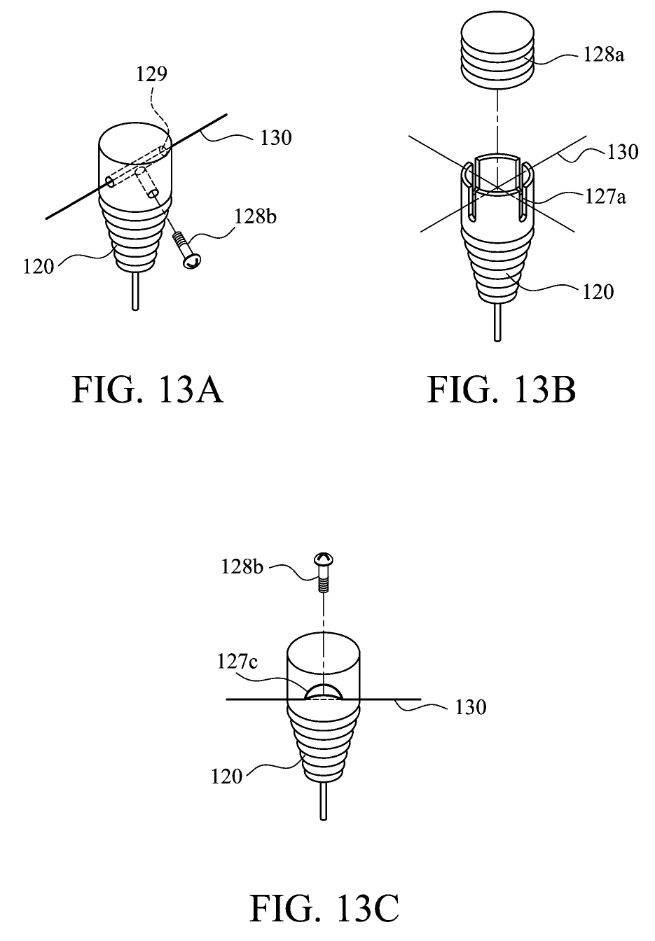

The connecting member 130 or the supporting member 110 is fixed on the anchoring member 120. The fixing method may be as follows: 1) the connecting member 130 passes through a hole 129 of the anchoring member 120 and the connecting member 130 is vertically fixed to the anchoring member 120 via an inner screw 128b; 2) the connecting member 130 is installed in and engaged with at least of groove 127a of the anchoring member 120 via the inner screw 128b; or 3) the connecting member 130 is engaged with a lateral groove 127c of the anchoring member 120 and the connecting member 130 is fixed to the anchoring member 120 via the inner screw 128b, a shown in FIGS. 13A, 13B and 13C. Connection between the connecting member 130, supporting member 110 and anchoring member 120 is complete fixation or dynamic connection. Dynamic connection is defined as having one end of the supporting member 110 and the connecting member 130 able to move, two ends of the supporting member 110 and the connecting member 130 able to move, three ends of the supporting member 110 and the connecting member 130 able to move or four ends of the supporting member 110 and the connecting member 130 able to move.

### Four Embodiment

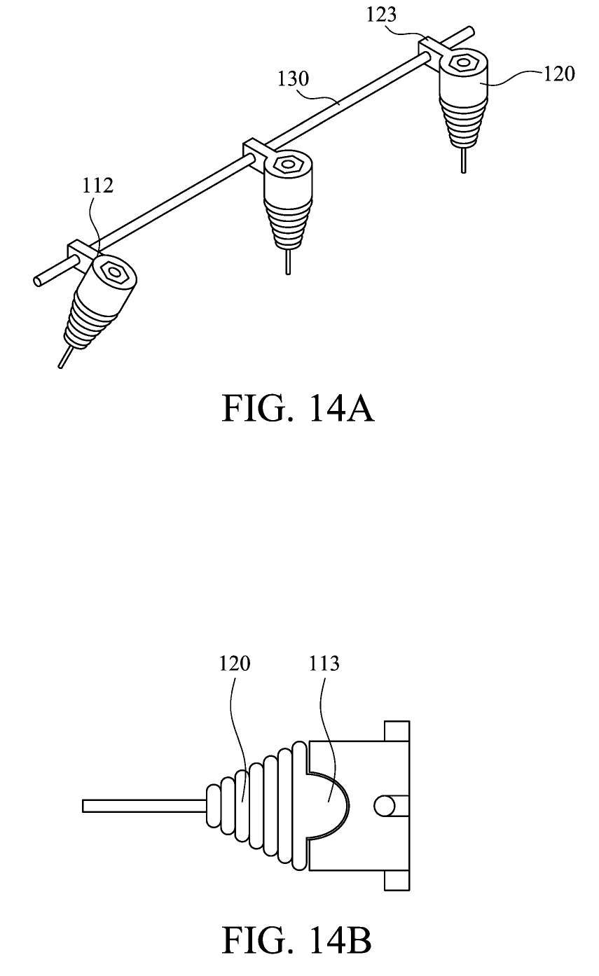

The anchoring member 120 comprises a lateral offset 123 to fix to the connecting member 130, and the lateral offset 123 is a joint 112. The joint 112 is completely or dynamically fixed. Dynamic connection is defined as having one end of the supporting member 110 and the connecting member 130 able to move, two ends of the supporting member 110 and the connecting member 130 able to move, three ends of the supporting member 110 and the connecting member 130 able to move or four ends of the supporting member 110 and the connecting member 130 able to move, as shown in FIG. 14A.

The anchoring member 120 comprises an inner joint 113 (shown in FIG. 14B).

The spinal dynamic stabilization device 100′ corrects degenerative spondylolisthesis and spinal stenosis and restores the original biological height between vertebras of the spine. The supporting member 110 is installed in the soft tissue ST which is supported and opens for supporting the adjacent spinous processes V1 (or vertebras V), as shown in FIG. 11. Two anchoring members 120 are fixed in (the vertebra bodies V4 of) the vertebra V which slid forward, for maintaining the relative position between the vertebra V which slid forward and another adjacent vertebra V. Similarly, two connecting members 130 respectively have suitable length to pull the vertebra V which slid forward back to the normal biological position. Similar to previous, the relative positions of the vertebra V which slid forward and another adjacent vertebra are fixed via two anchoring members 120 and two connecting member 130. Thus, the original biological height between the vertebras V can be restored. Further, dynamic stability between the vertebras V increases.

Similar to previous, for the spinal dynamic stabilization device, the supporting member is installed between the spinous processes of two adjacent vertebras for maintaining the original biological height between the vertebras. And, the supporting member provides a cushion for removing pain caused by nerve stress via the vertebras as a patient bends over backwards. The anchoring member and the connecting member correct spondylolisthesis and remove pain caused by nerve stress because of the spondylolisthesis. The spinal dynamic stabilization device decreases surgical complexity and time. Further, the spinal dynamic stabilization device does not require removal of bones, muscles and ligaments.

### Fifth Embodiment

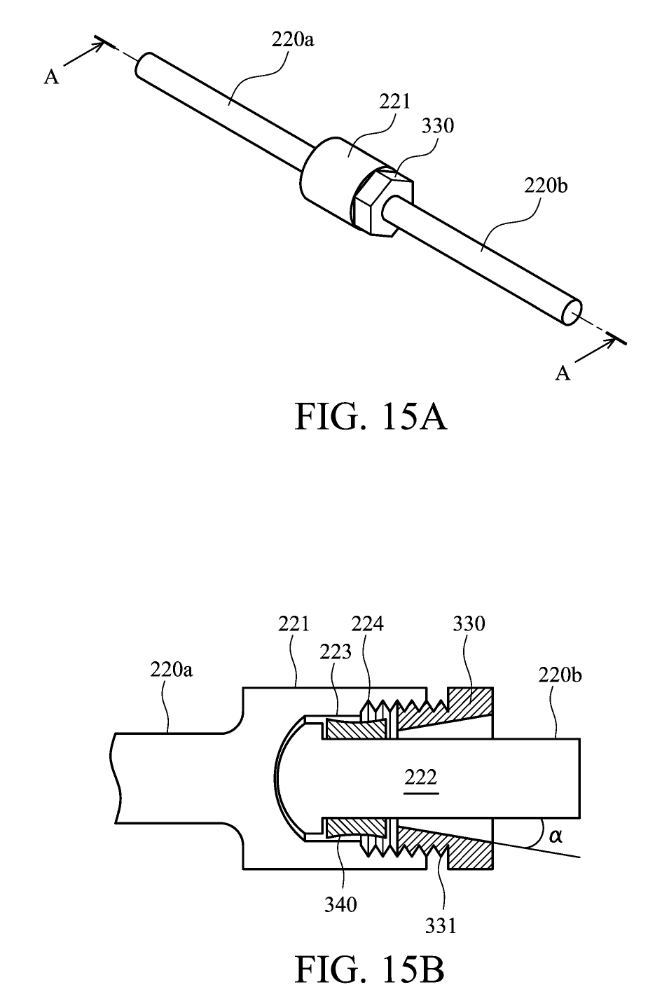

FIG. 15A is a schematic view of another embodiment of a connecting member and a clamp of a spinal dynamic stabilization device. FIG. 15B is a cross-sectional view along a cross-sectional line A-A. Referring to FIGS. 15A and 15B, the connecting members 220a and 220b are detachable. The connecting members 220a and 220b respectively comprise a first connecting end 221 and a second connecting end 222. The first connecting end 221 comprises a container 223 and a threading groove 224. The second connecting end 222 is disposed in the container 223. A fixing element 330 is a screw and comprises a threading portion 331 and a through hole 332. The connecting member 220b is inserted into the through hole 332 and then fixed to the connecting member 220a via threading portion 331 and the threading groove 224. Moreover, an elastic member 340 is installed between the first connecting end 221 and the second connecting end 222. Note that the container 223 comprises a concave portion 225, and the second connecting end 222 comprises a convex portion 226 connected to the concave portion 225. Note that the fixing element 330 comprises a taper angle α, and the taper angle α ranges from 2 to 12 degrees.

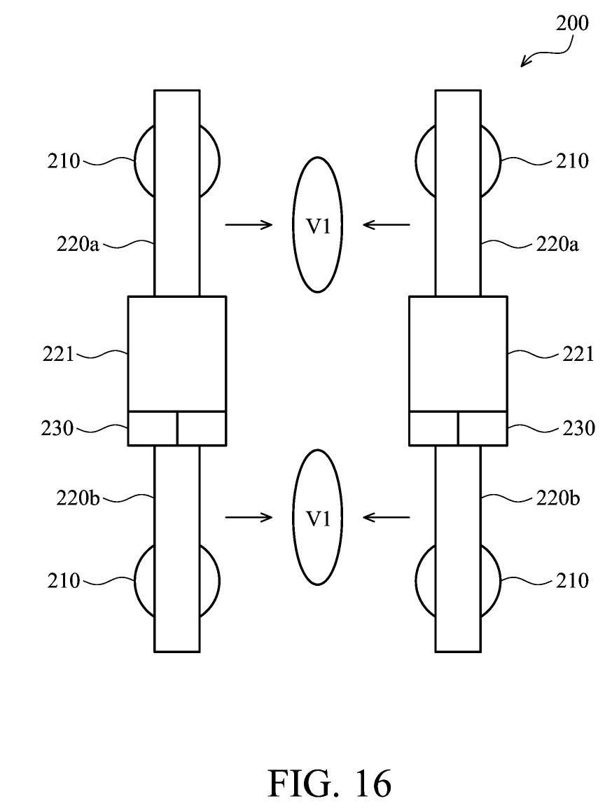

FIG. 16 is a schematic view showing another embodiment of a spinal dynamic stabilization device combined with a spine. The connecting members 220a and 220b and the fixing element 330 are fixed to the vertebral body V4 (shown in FIG. 5) via the anchoring member 210. Combination of the spinal dynamic stabilization device 200 and the vertebra bodies V4 is similar to FIG. 5, thus, it is omitted for brevity.

### Sixth Embodiment

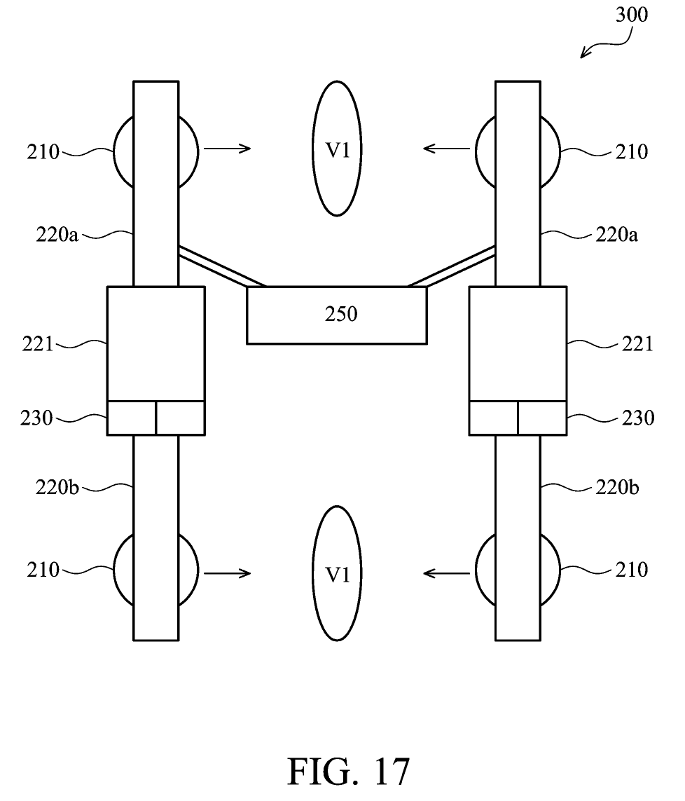

FIG. 17 is a schematic view showing another embodiment of a spinal dynamic stabilization device combined with a spine. The structure of the spinal dynamic stabilization device 300 is approximately similar to that in FIG. 16, thus, the similar structure is omitted for brevity. The difference is that the spinal dynamic stabilization device 300 further comprises a supporting member 250 connected between the connecting members 220a and assembled between the adjacent spinous processes V1.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A spinal dynamic stabilization device, maintaining an anatomical height between two adjacent vertebras, comprising:
a supporting member, disposed between the spinous processes;
at least an anchoring member, fixed in one of the vertebra; and
at least a connecting member, connecting the supporting member to the anchoring member,
wherein the connecting member, the supporting member, and the anchoring member relatively move and comprise at least a movable connecting point.

2. The spinal dynamic stabilization device as claimed in claim 1, wherein the supporting member is made of a rigid material, an elastic material, or a viscoelastic material, and the supporting member is assembled by a rigid mechanism, an elastic mechanism or a viscoelastic mechanism.

3. The spinal dynamic stabilization device as claimed in claim 3, wherein the structure of the supporting member comprises a solid structure, a hollow pillared structure, concentric circular structure, a mesh structure, a multi-layered structure, a radiate structure, or an artificial disc structure.

4. The spinal dynamic stabilization device as claimed in claim 1, wherein the supporting member is made of a bio-compatible material.

5. The spinal dynamic stabilization device as claimed in claim 1, wherein the connecting member is detachably connected to the supporting member.

6. The spinal dynamic stabilization device claimed in claim 1, wherein the connecting member and the supporting member are a single and unitary member.

7. The spinal dynamic stabilization device claimed in claim 1, wherein the supporting member comprises a spherical groove, the connecting member comprises a spherical member, and the spherical member is rotatably disposed in the spherical groove.

8. The spinal dynamic stabilization device claimed in claim 1, wherein the supporting member comprises a depression, the connecting member comprises a protrusion, and the protrusion is engaged with the depression.

9. The spinal dynamic stabilization device spinal dynamic stabilization device claimed in claim 1, wherein the supporting member comprises a groove and a screw hole, the connecting member comprises a connecting end and an opening, the screw hole adjoins the groove, the opening is disposed on the connecting end, the connecting end is disposed in the groove, the opening is adjusted and aligned according to the screw hole, and the connecting end is disposed in the opening via a screw and locked in the screw hole to fix the connecting end in the groove.

10. The spinal dynamic stabilization device claimed in claim 1, wherein the supporting member comprises a protrusion, the connecting member comprises a loop, and the loop is hooked onto the protrusion.

11. The spinal dynamic stabilization device claimed in claim 10, further comprising a clamp to fix and connect to the supporting member and the connecting member, wherein the clamp comprises a clipping groove and a clipping hole, the clipping groove is disposed on the outer side of the clipping hole, the clipping groove movably clips the connecting member, the supporting member is held in the clipping hole, and the connecting member and the supporting member form included angle.

12. The spinal dynamic stabilization device claimed in claim 1, wherein the connecting member is made of a rigid material, an elastic material, or a viscoelastic material, and the connecting member is assembled by a rigid mechanism, an elastic mechanism or a viscoelastic mechanism.

13. The spinal dynamic stabilization device claimed in claim 12, wherein the connecting member is linear shaped, pillared shaped, plate shaped, curve shaped or spring shaped.

14. The spinal dynamic stabilization device as claimed in claim 12, wherein the connecting member is made of a bio-compatible material.

15. The spinal dynamic stabilization device as claimed in claim 14, wherein the connecting member comprises a porous structure and a laminar structure.

16. The spinal dynamic stabilization device as claimed in claim 1, wherein the connecting member is detachably connected to the anchoring member.

17. The spinal dynamic stabilization device as claimed in claim 1, wherein the anchoring member comprises an outer thread, and the anchoring member is fixed in one of the vertebras.

18. The spinal dynamic stabilization device as claimed in claim 1, wherein the anchoring member is fixed in one of the vertebras via Polymethylmethacrylate (PMMA).

19. The spinal dynamic stabilization device as claimed in claim 1, wherein the anchoring member comprises a radiate hook, and the radiate hook is engaged in one of the vertebras.

20. The spinal dynamic stabilization device as claimed in claim 1, wherein the anchoring member is made of a bio-compatible material.

21. The spinal dynamic stabilization device as claimed in claim 1, wherein the anchoring member comprises a depression and a locking portion, the locking portion is installed adjacent to the depression, and the connecting member extends to the depression and is fixed in the anchoring member via the locking portion.

22. The spinal dynamic stabilization device as claimed in claim 1, wherein the connecting member comprises a loop, and the loop is hooked onto the anchoring member.

23. The spinal dynamic stabilization device as claimed in claim 1, wherein the anchoring member comprises a depression, the connecting member comprises an engaging portion, and the engaging portion is engaged with the depression.

24. The spinal dynamic stabilization device as claimed in claim 23, wherein the shapes of depression and the engaging portion are complementary.

25. The spinal dynamic stabilization device as claimed in claim 1, wherein the anchoring member comprises at least a groove, the groove is engaged with at least a connecting member, and the anchoring member comprises an inner groove to fix to an inner fixing device for fixing the connecting member.

26. The spinal dynamic stabilization device as claimed in claim 1, wherein the anchoring member comprises a lateral offset to fix to the connecting member, and the lateral offset is a joint.

27. The spinal dynamic stabilization device as claimed in claim 1, wherein the anchoring member comprises an inner joint.

28. The spinal dynamic stabilization device as claimed in claim 1, wherein when the number of the connecting member is plural, the connecting members respectively comprise a first connecting end with a container and a threading groove, and a second connecting end disposed in the container.

29. The spinal dynamic stabilization device as claimed in claim 28, further comprising a fixing element with a threading portion and a through hole, one of the connecting members is inserted into the through hole and then fixed to the other of the connecting members via threading portion 331 and the threading groove.

30. The spinal dynamic stabilization device as claimed in claim 29, wherein the fixing element comprises a taper angle, and the taper angle ranges from 2 to 12 degrees.
